(19) United States
(12) Patent Application Publication
(10) Pub. No.: US 2003/0147831 A1
(43) Pub. Date: Aug. 7, 2003

(54) Hair follicle growth

(76) Inventor: Olga Marko, Paramus, NJ (US)

Correspondence Address: FISH & RICHARDSON P.C., 45 ROCKEFELLER PLAZA, SUITE 2800, NEW YORK, NY, 10111, US

(21) Appl. No.: 10/330,584

(22) Filed: Dec. 27, 2002

Related U.S. Application Data
(60) Provisional application No. 60/345,902, filed on Dec. 31, 2001.

(51) Int. Cl. A61K 7/06; A61K 7/11; C12P 21/02

(52) U.S. Cl. 424/70.14; 435/69.1

(57) ABSTRACT

The invention provides compositions containing, and methods for making and using, a hair follicle growth factor produced by fat cells.

This application claims priority of U.S. Provisional Application No. 60/345,902, filed Dec. 31, 2001, the disclosure of which is incorporated herein by reference in its entirety.

### TECHNICAL FIELD

This invention relates to factors produced by fat cells, and more particularly to factors that promote the growth of hair.

### BACKGROUND

Baldness is a condition affecting a large proportion of the human male population and a significant proportion of the human female population. Currently used processes to treat baldness involve significant discomfort to the patient and/or have met with relatively limited success.

### SUMMARY

The roots of actively growing hairs are embedded in a layer of fat cells (adipocytes). The inventors noted that, while balding areas of the scalp are generally depleted of fat tissue, the occipital area of the scalp (in which balding seldom occurs) contains a thick layer of fat tissue. They considered it likely that fat cells produce a growth factor that is essential for hair growth. The experiments described below indicate this model to be correct.

The invention thus features a method of making a factor that stimulates hair growth. The method involves: (a) providing a population of cells comprising adipocytes, pre-adipocytes, or a mixture of adipocytes and pre-adipocytes; (b) culturing the population of cells; and (c) recovering the factor from the culture. The method can further comprise, prior to the culturing step, differentiating pre-adipocytes in the cell population into adipocytes.

Also provided by the invention is a method of treatment. The method involves: (a) identifying a subject having a region of skin in need of hair growth; and (b) administering to the region a composition comprising an isolated hair growth factor that is identical to a hair growth factor produced by adipocytes or pre-adipocytes.

Another aspect of the invention is an alternative method of treatment. The method involves: (a) identifying a subject having a region of skin in need of hair growth; and (b) administering to the region a composition comprising adipocytes, pre-adipocytes, or a mixture of adipocytes and pre-adipocytes.

Also embraced by the invention is a composition containing: (a) a hair growth factor that is identical to a hair growth factor produced by adipocytes or pre-adipocytes; and (b) a pharmaceutically acceptable carrier.

Also provided by the invention is a method of stimulating the growth of a hair. The method involves contacting the follicle of the hair with an isolated hair growth factor that is identical to a hair growth factor produced by adipocytes or pre-adipocytes. The contacting can be in vitro or the hair follicle can be in the skin of a mammalian subject, e.g., a human. The skin can be on the scalp of the human. In vivo contacting can be by administering to a subject a composition containing the isolated hair growth factor and, optionally, a pharmaceutically acceptable carrier.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., treating baldness, will be apparent from the following description and from the claims.

### DETAILED DESCRIPTION

The inventor has discovered that a growth factor produced by fat cells plays a role in the growth of hair. It is understood that such a growth factor can be a single molecular entity. Alternatively, it can be composed of multiple (e.g., two, three, four, five, six, seven, eight, nine, ten or more) molecular entities. Moreover such entities can be any biological molecules, e.g., protein, carbohydrate, lipid, nucleic acid, or a small molecule such as a vitamin or hormone (peptide or other). The factor can be used in a relatively crude form (e.g., as a culture supernatant), a semi-purified form, or a highly purified form. It will preferably be isolated.

Hair Growth Factor

An “isolated” factor as used herein refers to a factor which either has no naturally-occurring counterpart or has been separated or purified from components which naturally accompany it, e.g., in tissues such as skin, fat, pancreas, liver, spleen, ovary, testis, muscle, joint tissue, neural tissue, gastrointestinal tissue or tumor tissue, or body fluids such as blood, serum, or urine. Typically, the factor is considered “isolated” when it is at least 70%, by dry weight, free from the other naturally-occurring organic molecules with which it is naturally associated. Preferably, a preparation of a factor of the invention is at least 80%, more preferably at least 90%, and most preferably at least 99%, by dry weight, the factor of the invention. Thus, for example, a preparation of factor x is at least 80%, more preferably at least 90%, and most preferably at least 99%, by dry weight, factor x. Since a factor that is chemically synthesized is, by its nature, separated from the components that naturally accompany it, the synthetic factor is “isolated.”

An isolated factor of the invention can be obtained, for example, by extraction from a natural source (e.g., from tissues); by, in the case of a polypeptide, expression of a recombinant nucleic acid encoding the polypeptide; or by chemical synthesis. A factor that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

With respect to polypeptide hair growth factors produced by adipocytes and/or pre-adipocytes, the invention includes full-length immature (unprocessed) polypeptides, full-length mature polypeptides, and functional fragments of either. “Polypeptide” and “protein” are used interchangeably and mean any peptide-linked chain of amino acids, regardless of length or post-translational modification. As used herein, a “functional fragment” of a hair growth polypeptide is a fragment of the full-length, wild-type, mature hair growth polypeptide that is shorter than the full-length, wild-type, mature hair growth polypeptide but has at least 20% (e.g., at least: 20%; 30%; 40%; 50%; 60%; 70%; 80%; 85%; 90%; 95%; 98%; 99%; 99.5%; 99.8%; 100%; or even more) of the hair growth promoting activity of the full-length, wild-type, mature hair growth polypeptide.

The invention also features the hair growth polypeptides, or functional fragments thereof, with not more than 25 (e.g., not more than; 25; 20; 15; 12; 10; nine; eight; seven; six; five; four; three; two; or one) conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. A polypeptide (including a functional fragment) with one or more conservative substitutions should have at least 20% (as above) of the hair growth promoting activity of the corresponding parent, unmutated polypeptide.

The polypeptides of the invention can be purified from natural sources (e.g., blood, serum, plasma, tissues or cells such as adipocytes or pre-adipocytes). Smaller peptides (less than 50 amino acids long) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals (see below). See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention also include those described above, but modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected peptide. The peptide motif provides the peptidomimetic compound with the ability to stimulate hair growth in a manner qualitatively identical to that of the hair growth polypeptide functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

The invention also provides nucleic acid molecules encoding the above-described hair growth polypeptides. The nucleic acid molecules of the invention can be cDNA, genomic DNA, synthetic DNA, or RNA, and can be double-stranded or single-stranded (i.e., either a sense or an antisense strand). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide. In addition, these nucleic acid molecules are not limited to coding sequences, e.g., they can include some or all of the non-coding sequences that lie upstream or downstream from a coding sequence.

The nucleic acid molecules of the invention can be synthesized (for example, by phosphoramidite-based synthesis) or obtained from a biological cell, such as the cell of a mammal. The nucleic acids can be those of a human, non-human primate (e.g., monkey), mouse, rat, guinea pig, cow, sheep, horse, pig, rabbit, dog, or cat. Combinations or modifications of the nucleotides within these types of nucleic acids are also encompassed by the invention.

In addition, the isolated nucleic acid molecules of the invention encompass segments that are not found as such in the natural state. Thus, the invention encompasses recombinant nucleic acid molecules incorporated into a vector (for example, a plasmid or viral vector) or into the genome of a heterologous cell (or the genome of a homologous cell, at a position other than the natural chromosomal location).

Techniques associated with detection or regulation of genes are well known to skilled artisans. Such techniques can be used to diagnose and/or treat disorders associated with aberrant hair growth polypeptide expression, e.g., baldness.

Hybridization can also be used as a measure of homology between two nucleic acid sequences. A hair growth polypeptide-encoding nucleic acid sequence, or a portion thereof, can be used as a hybridization probe according to standard hybridization techniques. The hybridization of a hair growth polypeptide nucleic acid probe to DNA or RNA from a test source (e.g., a mammalian cell) is an indication of the presence of the hair growth polypeptide-encoding DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2×sodium chloride/sodium citrate (SSC) at 30° C., followed by a wash in 1×SSC, 0.1% SDS at 50° C. Highly stringent conditions are defined as equivalent to hybridization in 6×sodium chloride/sodium citrate (SSC) at 45° C., followed by a wash in 0.2×SSC, 0.1% SDS at 65° C.

The invention also encompasses: (a) vectors (see below) that contain any of the foregoing hair growth polypeptide coding sequences and/or their complements (that is, “antisense” sequences); (b) expression vectors that contain any of the foregoing hair growth polypeptide coding sequences operably linked to any transcriptional/translational regulatory elements (examples of which are given below) necessary to direct expression of the coding sequences; (c) expression vectors encoding, in addition to a hair growth polypeptide, a sequence unrelated to the hair growth polypeptide, such as a reporter, a marker, or a signal peptide fused to the hair growth polypeptide; and (d) genetically engineered host cells (see below) that contain any of the foregoing expression vectors and thereby express the nucleic acid molecules of the invention. As used herein, “operably linked” means incorporated into a genetic construct so that expression control sequences effectively control expression of a coding sequence of interest.

Recombinant nucleic acid molecules can contain a sequence encoding hair growth polypeptide or the hair growth polypeptide having a heterologous signal sequence. The full-length hair growth polypeptide, or a fragment thereof, may be fused to such heterologous signal sequences or to additional polypeptides, as described below. Similarly, the nucleic acid molecules of the invention can encode the mature form of the hair growth polypeptide or a form that includes an exogenous polypeptide that facilitates secretion.

The transcriptional/translational regulatory elements referred to above include but are not limited to inducible and non-inducible promoters, enhancers, operators and other elements that are known to those skilled in the art and that drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Similarly, the nucleic acid can form part of a hybrid gene encoding additional polypeptide sequences, for example, a sequence that functions as a marker or reporter. Examples of marker and reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being a hair growth polypeptide and the second portion being, for example, the reporter described above or an Ig constant region or part of an Ig constant region, e.g., the CH2 and CH3 domains of IgG2 a heavy chain. Other hybrids could include an antigenic tag or His tag to facilitate purification.

The expression systems that may be used for purposes of the invention include but are not limited to microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecule of the invention; insect cell systems infected with recombinant virus expression vectors (for example, baculovirus) containing a nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing a hair growth polypeptide-encoding nucleotide sequence; or mammalian cell systems (for example, COS, CHO, BHK, 293, VERO, HeLa, MDCK, W138, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

Use of a Hair Growth Factor

The growth factor can be utilized in many different ways. For example, it can be a component of an injectable composition which is injected into a balding area (e.g., the scalp). Whether provided dry or in solution, the compositions of the invention can be prepared for storage by mixing them with any one or more of a variety of pharmaceutically acceptable carriers, excipients or stabilizers known in the art [Remington's Pharmaceutical Sciences, 16th Edition, Osol, A. Ed. 1980]. Acceptable carriers, excipients, or stabilizers are nontoxic to recipients at the dosages and concentrations employed, and include: buffers, such as phosphate, citrate, and other non-toxic organic acids; antioxidants such as ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol, or sorbitol; salt-forming counterions such as sodium; and/or nonionic surfactants such as Tween, Pluronics, or PEG. Alternatively, the factor can be a component of a cream or solution to be applied topically to a balding area (e.g., scalp), optionally in combination with any known non-toxic delivery agent and/or penetrant.

The compositions of the invention can be administered orally or by intravenous infusion, or injected subcutaneously, intramuscularly, intrathecally, intraperitoneally, intrarectally, intravaginally, intranasally, intragastrically, intratracheally, or intrapulmonarily. The dosage required depends on the choice of the route of administration; the nature of the formulation; the nature of the patient's condition; the subject's size, weight, surface area, age, and sex; other drugs being administered; and the judgment of the attending physician. Suitable dosages are in the range of 0.01-100.0 mg/kg. Wide variations in the needed dosage are to be expected in view of the differing efficiencies of various routes of administration. Variations in these dosage levels can be adjusted using standard empirical routines for optimization as is well understood in the art. Administrations can be single or multiple (e.g., 2-, 3-, 4-, 6-, 8-, 10-, 20-, 50-,100-, 150-, or more fold). Encapsulation of the polypeptide in a suitable delivery vehicle (e.g., polymeric microparticles or implantable devices) may increase the efficiency of delivery, particularly for oral delivery.

Furthermore, the factor can be a component of a composition (e.g., a fluid, gel, or solid composition) also containing hair follicle cells, e.g., dermal papillae cells, outer and inner root shaft cells such as keratinocytes and fibroblasts. Such compositions can be injected into balding areas (e.g., scalp) of a patient. To obtain follicle cells, 3 to 6 mm punch biopsies of skin obtained from the occipital area of the same subject (or another subject), where there is healthy hair growth, and individual healthy hair follicles can be isolated from them. From the isolated hair follicles, their cellular components can be obtained and grown in vitro. Follicle cells include dermal papilla cells, outer shaft epithelial cells, and inner root fibroblastic cells as well. Inner and outer shaft cells can be isolated from the hair follicles. Alternatively, skin keratinocytes and skin fibroblasts obtained from a skin biopsy can be used. Such cells are known to adapt to new environments. Generally the cells to be injected will not be of one type only. Preferably the compositions will contain cells of all three types. The compositions can also contain additional growth factors known to promote growth of hair; such factors include, without limitation, insulin, insulin-like growth factor (IGF), interleukin-4 (IL-4), transforming growth factor (TGF) (e.g., TGFα or TGFβ1), basic fibroblast growth factor (bFGF), epidermal growth factor (EGF), platelet-derived growth factor (PDGF), or biotin. Such a process could significantly cut the risk factor of hair transplant, trauma and financial burden of the individual. This process could be carried out in a doctor's office without the recovery time of in-patient surgery and the patient could go back to work the same day.

Alternatively, a polynucleotide containing a nucleic acid sequence encoding a hair growth polypeptide or functional fragment thereof can be delivered to cells in a mammalian subject. Expression of the coding sequence can be directed to any cell in the body of the subject but will preferably be directed to cells in, or in the vicinity of, hair follicles (e.g., cells of the dermis). Uptake of nucleic acids by cells can be achieved by, for example, the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al. (1995), J. Mol. Med. 73, 479]. Alternatively, tissue specific targeting can be achieved by the use of tissue-specific transcriptional regulatory elements (TRE) which are known in the art. Delivery of “naked DNA” (i.e., without a delivery vehicle) to an intramuscular, intradermal, or subcutaneous site is another means to achieve in vivo expression.

In the relevant polynucleotides (e.g., expression vectors), the nucleic acid sequence encoding the hair growth polypeptide or functional fragment of interest with an initiator methionine and optionally a targeting sequence is operatively linked to a promoter or enhancer-promoter combination.

Short amino acid sequences can act as signals to direct proteins to specific intracellular compartments. Such signal sequences are described in detail in U.S. Pat. No. 5,827,516, incorporated herein by reference in its entirety.

Enhancers provide expression specificity in terms of time, location, and level. Unlike a promoter, an enhancer can function when located at variable distances from the transcription initiation site, provided a promoter is present. An enhancer can also be located downstream of the transcription initiation site. To bring a coding sequence under the control of a promoter, it is necessary to position the translation initiation site of the translational reading frame of the peptide or polypeptide between one and about fifty nucleotides downstream (3′) of the promoter. The coding sequence of the expression vector is operatively linked to a transcription terminating region.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., decreased proliferation of cancer cells) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

An ex vivo strategy can involve transfecting or transducing cells obtained from the subject with a polynucleotide encoding a hair growth polypeptide or functional fragment-encoding nucleic acid sequences. The transfected or transduced cells are then returned to the subject. The cells can be any of a wide range of types including, without limitation, hemopoietic cells (e.g., bone marrow cells, macrophages, monocytes, dendritic cells, T cells, or B cells), fibroblasts, epithelial cells, endothelial cells, keratinocytes, or muscle cells. They can also be any of the hair follicle cells recited herein. Such transfected or transduced cells act as a source of the hair growth polypeptide or functional fragment for as long as they survive in the subject.

The ex vivo methods include the steps of harvesting cells from a subject, culturing the cells, transducing them with an expression vector, and maintaining the cells under conditions suitable for expression of the hair growth polypeptide or functional fragment. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells can then be lethally irradiated (if desired) and injected or implanted into the patient.

The growth factor can also be used, optionally with other factors, as a culture medium supplement for in vitro growth and maintenance of hair follicles. The tissue culture techniques described below (and variations of them that would be obvious to those in the art) can be used to preserve hair follicles in culture for prolonged periods of time, e.g., for autologous or allogeneic transplantation not performed on the day of collection. The hair growth factor and/or hair follicles grown in culture can be used in basic scientific studies on hair biology. The factor can also be used as a “positive control” in in vitro assays of hair growth.

The invention also includes a method of treating baldness by injection into a subject's balding area (e.g., scalp) of fat cells (e.g., adipocytes or pre-adipocytes), preferably (but not necessarily) obtained from the same patient. Such cells can be freshly harvested from a donor or cultured prior to administration to the patient. The fat cells can be injected with hair follicles, hair follicle cells (see above), and/or the described hair follicle growth factor. The fat cells will preferably be more than 10% (e.g., more than 10%, more than 15%, more than 20%, more than 30%, more than 40%, more than 50%, more than 60%, more than 70%, more than 80%, more than 90%, more than 95%, more than 98%, more than 99%, more than 99.5%) or 100% adipocytes and/or pre-adipocytes.

While hair follicles to be treated by the methods of the invention will generally be in the skin on the scalp of a subject, such skin can be any in any part of the body. It could be, without limitation, on the face, torso, back, abdomen, arms, leg, axilla, or pubic area of a subject.

Methods of Generating and Growing Fat Cells and Making Hair Growth Factors

The invention also features processes for recovering healthy hair follicles from a skin biopsy and methods for in vitro growth of and differentiation to adipocytes from pre-adipocytes from bone marrow and fat tissue such as human bone marrow or fat tissue.

Also embraced by the invention are methods of growing adipocytes, pre-adipocytes, hair follicles, or hair follicle cells. Growth of such cells can be by, for example, the methods disclosed herein or in plasma clots (e.g., plasma clots produced from a patients own plasma). To these clots autologous or allogeneic fibroblasts (e.g., proliferation-inhibited fibroblasts) can be added. Proliferation-inhibited fibroblasts do not grow but produce exogenous growth factors that enhance viability and growth of, e.g., hair follicles or hair follicle cells in vitro for greater than 6 months. Naturally, the growth media (including plasma clots) used for growing hair follicles and/or hair follicle cells can be supplemented with a source of the above-described fat cell-derived hair follicle growth factor and/or any of the hair follicle growth stimulating factors disclosed herein.

The invention also features methods of making a fat cell (e.g., adipocyte and/or pre-adipocyte)-derived hair follicle growth factor. Such methods include culturing of adipocyte- and/or pre-adipocyte-containing cell populations for sufficient time to obtain a desired level of hair follicle growth promoting activity (measured, for example, as described herein) in the cells and/or in culture supernatants of the cells. The cultures can contain unpurified adipocytes and/or pre-adipocytes but will preferably contain more than 10% (e.g., more than 10%, more than 15%, more than 20%, more than 30%, more than 40%, more than 50%, more than 60%, more than 70%, more than 80%, more than 90%, more than 95%, more than 98%, more than 99%, more than 99.5%) or 100% adipocytes and/or pre-adipocytes. When the cells are producing the desired level of activity, the culture supernatants are isolated from the cells and/or cell lysates are prepared from the cells by methods described herein or by any of a variety of methods known in the art. The supernatants and/or lysates can be used without further purification as a source of hair follicle or hair follicle cell growth promoting activity in any of the methods of the invention. Alternatively, the hair follicle or hair follicle cell growth promoting factor can be semi-purifed or highly purified from culture supernatants and/or cell lysates prior to such use.

All cell types, hair follicles, and patients referred to above can be of any mammalian species, e.g., human, non-human primates, horses, cats, dogs, cattle, goats, sheep, rabbits, mice, rats, guinea pigs, or hamsters.

The following examples serve to illustrate, not limit, the invention.

### EXAMPLES

### Example 1

### Establishment of Pre-Adipocyte Cell Lines from Rat Bone Marrow

Rat and human pre-adipocyte cell lines were derived by differentiating bone marrow precursor cells (of pre-adipocytes) in rat and human bone marrow into pre-adipocytes in culture. Moreover, these culture-differentiated pre-adipocytes could be further differentiated in vitro to multilocular adipocytes (as assessed histologically) resembling the cells of brown adipose tissue. In initial experiments, a rat pre-adipocyte cell line was used to produce adipocytes which were used as a source of the hair growth factors described herein [Marko et al. (1995) Endocrinology, 136: 4582-4588; incorporated herein by reference in its entirety].

Rat bone marrow-derived pre-adipocyte cell lines were established as follows. Bone marrow was obtained by syringe aspiration of rat limb bones (e.g., femurs) and the isolated bone marrow cells were cultured for 4 days in Dulbecco's Modified Eagle's Medium (DMEM) containing a low concentration of glucose (1000 mg/L), sodium pyruvate (110 mg/ml), L-glutamine (2 mM) and heat-inactivated fetal bovine serum (FBS) (10%). Cells not adhering to the plastic tissue culture vessel (e.g. tissue culture flask, well, or dish) (“non-adherent cells”) were removed and fresh culture medium was added to the “adherent cells” (cells adhering to any of the above plastic tissue culture vessels). Stromal fibroblast-like cells were observed in the cultures at this time. The cultures were supplemented with human umbilical vein endothelial cell (HUVEC)-conditioned medium (added to the cultures at a final concentration of about 20%) for one month. The HUVEC-conditioned medium was prepared by growing the HUVEC in KGM culture medium (Clonetics, San Diego, Calif.). Every three days, culture medium was removed from the HUVEC and was used as a source of HUVEC-conditioned medium. After removal of the medium, fresh KGM medium was added to the HUVEC.

Growth of the bone marrow-derived cells in the HUVEC-conditioned medium led to an increase in the proportion of epitheloid-like cells. Fibroblast growth was attenuated by growing the cells in low calcium (0.5 mM) containing medium (KGM medium, Clonetics). Stromal fibroblasts were found to be more readily detachable from the culture vessel bottoms by “mild” trypsinization than the epitheloid-like cells. Thus, the cultures were enriched for epitheliod-like cells by treating the adherent cells with trypsin-EDTA. Prior to trypsinization, the culture medium was completely removed and the adherent cells were washed once with phosphate buffered saline (PBS) without calcium and magnesium and twice with trypsin (0.05%; w:v)-EDTA (5.3 mM). After the last wash in trypsin-EDTA, the cells were incubated in residual trypsin-EDTA at room temperature for 1-3 minutes (i.e., until the fibroblast-like cells “rounded-up” and detached from the plastic bottom of the tissue culture flask). The non-adherent cells were removed and the remaining adherent cells were allowed to grow for several days in culture medium added to the tissue culture flasks. This enrichment process was repeated several times until the epitheliod-like (i.e., no-fibroblastic) cells constituted the majority of cells in the culture. At this point, the cells were allowed to grow until sufficient cells for subsequent enrichment steps were obtained.

Next, a Ficoll or Percoll density gradient system was used to enrich for the epitheloid-like cells. The cells were detached from the flasks by exposure to trypsin (0.05%)-EDTA (5.3 mM) for sufficient time to detach all the cells adhering to the bottom of the tissue culture flask. Where Ficoll was used, the cells (3-5×10⁶ in 10 ml of culture medium) were layered on top of a Ficoll gradient consisting of 3 ml of a 1:1 mixture of lymphocyte separation medium (Organon Teknika Corp., Durham, N.C.) and DMEM which had, in turn, been layered above 3 ml of undiluted lymphocyte separation medium in a centrifuge tube. The centrifuge tube was centrifuged for 30 minutes at room temperature at 2,400 rpm. Cells banding at the lower gradient interface (i.e., at the interface of the diluted and the undiluted lymphocyte separation medium) were plated in tissue culture medium (DMEM containing glucose (1000 mg/L), L-glutamine (2 mM), sodium pyruvate (110 mg/ml), penicillin-streptomycin solution (100 U/ml), heat inactivated FBS (2.5%), recombinant human acidic fibroblast growth factor (aFGF; 2.5 ng/ml), and heparin (5 μg/ml)). Human aFGF was found to be as active on rat cells as rat aFGF. The cultures were further enriched for eptheloid-like cells by differential trypsinization (as described above) and differential seeding. Differential seeding involved seeding into a tissue culture dish, incubating the dish at 36.5° C. for 5 minutes, and removing the unattached cells. The process was repeated with the unattached cells. It was performed again with the unattached cells recovered after the second incubation and, in some experiments, again with unattached cells recovered after the third incubation. The attached populations from all steps were retained and expanded in culture. A population containing substantially pure pre-adipocytes was obtained at passage 10 after several cycles of the enrichment procedures described above. After differentiation of such lines into adipocytes (see below), the relevant cultures contained 95 to 100% adipocytes. These cells were grown continuously in DMEM containing glucose (1,000 mg/liter), L-glutamine (2 mM), sodium pyruvate (110 mg/liter), penicillin-streptomycin (100 U/ml), heat inactivated FBS (2.5%), recombinant human acidic fibroblast growth factor (aFGF; 2.5 ng/ml), and heparin (5 μg/ml). In the absence of aFGF, a small proportion of the cells was observed to spontaneously differentiate into adipocytes. Cultures were never allowed to grow to confluence.

Clonal populations of cells were obtained by seeding cells at very low numbers into plasma clots and allowing the cells to grow and form discrete colonies in the clots. Individual colonies were picked out of the clots with fine Pasteur pipettes and grown up.

### Example 2

### Differentiation of Bone-Marrow Derived Pre-Adipocytes into Adipocytes

Adipocytes were obtained from the above bone marrow-derived pre-adipocyte lines as follows. Cells harvested from the cultures were seeded at a density of about 8×10³/cm². Forty eight hours after seeding, the cells reached a density of about 2-3×10⁴ cells/cm². The culture medium was replaced with fresh medium (DMEM containing glucose (1,000 mg/L), sodium pyruvate (100 mg/ml), glutamine (2 mM), penicillin-streptomycin (100 U/ml), heat inactivated FBS (10%), insulin (5 μg/ml), isobutyl methyl xanthine (IBMX; 0.5 mM) and dexamethasone 21-phosphate disodium salt (0.25 μM)). After 48 hours of culture, this medium was replaced with DMEM containing glucose (1,000 mg/L), sodium pyruvate (100 mg/L), glutamine (2 mM) and penicillin-streptomycin (100 U/ml), and heat inactivated FBS (5%)) (“standard culture medium”). The medium in the cultures was replaced with fresh standard culture medium every 3-4 days. 8-15 days after transfer to standard culture medium, the cultures contained 95-100% fully differentiated adipocytes.

### Example 3

### Supernatants and Lysates of Bone Marrow-Derived Adipocytes Promote Growth of Hair

Culture supernatants and lysates of adipocytes derived by differentiation of the above described rat bone marrow-derived pre-adipocyte cell lines were tested for growth-promoting activity on human hair follicles isolated as described below. Test supernatants were prepared by adding fresh medium to the cultures of the above-described rat bone marrow-derived, fully differentiated adipocytes in either T-75 or T-150 tissue culture flasks. T-75 flasks contained approximately 20 ml of culture medium and T-150 flasks contained about 40 ml of culture medium. After 3-4 days of culture, the medium was removed, separated from any non-adherent cells by centrifugation, and sterile filtered. At the time of recovery of the culture supernatants, the T-75 tissue culture flasks contained about 3×10⁶ to about 5×10⁶ cells and the T-150 tissue culture flasks contained about 5×10⁶ to about 9×10⁶ cells.

Adipocyte lysates were prepared by rapidly freezing and thawing the cells harvested from the cultures (with a rubber policeman) in the culture medium used for hair follicle growth (see below). Lysis was carried out at cell concentration of about 1×10⁶ cells/ml of culture medium. Cell debris, aggregated proteins, and released fat were removed by centrifugation and liquid phase was tested for hair follicle growth-promoting activity.

The conditioned culture medium was tested at a final concentration of 20%. In assays similar to those described below for testing supernatants from cultures of human fat fragments, the culture supernatants from the rat bone marrow-derived adipocytes were found to stimulate growth of hair in vitro. The changes were observed within 48-72 hours of initiating the cultures and were manifested by hair growth in the range of about 3 to about 5 mm in length. This activity was detected also in the adipocyte lysates; However the activity was lower than that detected in the adipocyte culture supernatants. In control cultures not containing conditioned medium or cell lysate, no significant hair growth was seen.

### Example 4

### Production of Hair Growth Factor by Human Fat Tissue

Since human fat is readily obtainable during surgery, the inventors have used human fat tissue from sources such as thigh, abdomen, scalp, eye lid, and face for experimentation. The source of the fat is not limited to any particular the body location. Fat was separated from membrane and dermal components and small fat fragments (approximately cubic in shape with each dimension being about 3-5 mm) were placed into tissue culture vessels. Cultures were performed in DMEM containing glucose (4,500 mg/L), L-glutamine (2 mM), gentacmicin (10 μg/ml), heat inactivated FBS (2.5%), recombinant human aFGF (5 ng/ml), and heparin (5 μg/ml). The fat fragments actively metabolized and shed cells with the morphology of pre-adipocytes. The cells showed mitochondrial activation with a low proportion (about 5%- about 15%) of the cells spontaneously differentiating into adipocytes. If culture medium without aFGF was used, rapid fibroblast growth was observed.

The fat fragments were maintained in culture for more than a year. Throughout this period, pre-adipocytes continued to be shed from the fragments and the pre-adipocytes proliferated in the cultures. The fat fragments were repeatedly passaged into fresh tissue culture flasks. Medium harvested from the cultures containing the fat tissue and the pre-adipocyte cells was tested for growth-promoting activity on isolated human hair follicles. This conditioned medium exhibited essentially the same effect as the above-described supernatant of rat adipocytes differentiated from bone-marrow derived pre-adipocytes.

Following the same procedure described above for rat bone marrow, a number of human pre-adipocyte lines were also established from human bone marrow. The culture medium used was DMEM containing glucose (4,500 mg/L), L-glutamine (2 mM), gentacmicin (10 μg/ml), heat inactivated FBS (2.5%), recombinant human aFGF (5 ng/ml), and heparin (5 μg/ml).

### Example 5

### Culture Supernatants of Human Fat Fragments Promote Hair Growth

Culture supernatants from the cultures of human fat fragments and pre-adipocytes were tested in vitro for growth promoting activity with both isolated hair follicles as well with skin fragments obtained from balding scalp. Isolated hair follicles were obtained by cutting human scalp tissue into approximately cubic fragments with each dimension being about 2-3 mm. The upper epidermis was removed and discarded, leaving dermal and fat intact. After culture of these fragments for 24-72 hours, the tissue softened and intact individual follicles could be removed with forceps. Hair follicles were also isolated by dissecting them directly from the scalp tissue. In the experiments with the isolated hair follicles, growth of about 3-5 mm of the inner hair shaft was observed in hair follicle cultures containing conditioned medium after 48-72 hours in culture. No visible effect on the hair follicles was seen in control cultures without conditioned medium.

The fragments of balding scalp were tested in a transwell culture system for susceptibility to hair follicle growth promotion by the pre-adipocyte culture supernatant. In the transwell system the balding scalp fragments were placed on one side of a semi-permeable membrane and the pre-adipocyte conditioned medium on the other side of semi-permeable membrane with a pore size of 0.22 μ. Conditioned medium was used at a final concentration of 20% (based on the total volume of medium on both sides of the semi-permeable membrane) for both initiation of the cultures and for medium changes which occurred twice per week. The culture medium in which the conditioned medium was diluted and which was used throughout the culture period was DMEM containing D-glucose (4,500 mg/l), L-glutamine (2 mM), heat inactivated FBS, recombinant aFGF (5 ng/ml), heparin (5 μg/ml) and gentamicin (10 μg/ml). Within 48 hours of initiating cultures containing the balding scalp samples and conditioned medium, thickening of the epidermis in some areas of the scalp sample was observed, with hair growth occurring 5-7 days later. Neither of these events occurred in cultures not containing conditioned medium.

A significant improvement in survival of hair follicles and growth in the presence of pre-adipocytes and dermal fibroblasts was also observed in separate experiments.

In the above described methods of producing the hair growth factor of the invention, instead of recovering culture supernatant as a source of growth factor, the factor could also be recovered as an cell extract of the cultured cells, e.g., as a cell lysate.

A number of embodiments of the invention have been described. Nevertheless, it will be understood that various modifications may be made without departing from the spirit and scope of the invention. Accordingly, other embodiments are within the scope of the following claim.

What is claimed is:

1. A method of making a factor that stimulates hair growth, the method comprising:
(a) providing a population of cells comprising adipocytes, pre-adipocytes, or a mixture of adipocytes and pre-adipocytes;
(b) culturing the population of cells; and
(c) recovering the factor from the culture.

2. The method of claim 1, further comprising, prior to the culturing step, differentiating pre-adipocytes in the cell population into adipocytes.

3. A method of treatment comprising:
(a) identifying a subject having a region of skin in need of hair growth; and
(b) administering to the region a composition comprising an isolated hair growth factor that is identical to a hair growth factor produced by adipocytes or pre-adipocytes.

4. A method of treatment comprising:
(a) identifying a subject having a region of skin in need of hair growth; and
(b) administering to the region a composition comprising adipocytes, pre-adipocytes, or a mixture of adipocytes and pre-adipocytes.

5. A composition comprising:
(a) a hair growth factor that is identical to a hair growth factor produced by adipocytes or pre-adipocytes; and
(b) a pharmaceutically acceptable carrier.

6. A method of stimulating the growth of a hair, the method comprising contacting the follicle of the hair with an isolated hair growth factor that is identical to a hair growth factor produced by adipocyte or pre-adipocytes.

7. The method of claim 6, wherein the contacting is in vitro.

8. The method of claim 6, wherein the hair follicle is in the skin of a mammalian subject.

9. The method of claim 8, wherein the mammalian subject is a human.

10. The method of claim 9, wherein the skin is on the scalp of the human.

11. The method of claim 8, wherein the contacting comprises administering to the subject a composition comprising the isolated hair growth factor.
